(19) United States
(12) Patent Application Publication
(10) Pub. No.: US 2010/0222647 A1
(43) Pub. Date: Sep. 2, 2010

(54) SYSTEM AND METHOD FOR ANALYSIS AND TREATMENT OF A BODY TISSUE

(75) Inventors: Dan Hashimshony, Givat Ada (IL); Gil Cohen, Jerusalem (IL)

Correspondence Address: BROWDY AND NEIMARK, P.L.L.C.;624 NINTH STREET, NW, SUITE 300, WASHINGTON, DC, 20001-5303, US

(73) Assignee: Dune Medical Devices Ltd., Caesarea (IL)

(21) Appl. No.: 12/161,431

(22) PCT Filed: Jan. 18, 2007
(86) PCT No.: PCT/IL07/00071

§ 371 Date: Jul. 18, 2008

Related U.S. Application Data
(60) Provisional application No. 60/759,555, filed on Jan. 18, 2006.

(51) Int. Cl. A61B 5/055; A61B 5/00

(52) U.S. Cl. 600/301; 600/411

(57) ABSTRACT

The invention provides a system and method for analysis and treatment of a tissue site. The system of the invention includes a probe unit containing one or more tissue sensing and monitoring probes configured to measure one or more parameters indicative of one or more states of the tissue site and one or more tissue treatment probes configured to deliver a treatment to the tissue site. A processor receives signals from the sensing and monitoring probes and determines whether the probe unit is located at the tissue site to be treated. The treatment and monitoring probes are activated in order to monitor the state of the tissue site while the treatment is being delivered to the tissue site. The processor receives signals from the sensing and monitoring probes during delivery of the treatment indicative of the state of the tissue site and determines, as the treatment is being carried out, any one or more of whether the treatment carried out so far is adequate, whether an additional treatment needs to be carried out, and whether the parameters of the treatment or the treatment targets need to be modified.

### FIELD OF THE INVENTION

This invention relates to medical systems and methods, and more specifically to such systems and methods for performing a medical procedure.

### BACKGROUND OF THE INVENTION

Systems for analyzing a tissue in order to determine whether the tissue is in need of a treatment are known in the art. Similarly, systems for providing a treatment to a tissue in need of such treatment are also known. Such systems are described in the following publications.

U.S. Pat. No. 6,711,429 describes a system and method for recording and displaying, in an image of the body the location of a point-of-interest in the body during a medical procedure. The method involves (a) establishing the location in the body of the point-of-interest; (b) inserting a catheter, including a first location implement, into the body; (c) obtaining an image of a portion of the body; (d) establishing a location of the imaging instrument in the body; (e) advancing the catheter to the point-of-interest and, using a locating implement, recording the location of the point-of-interest; and (f) displaying and highlighting the point-of-interest in image. In course of the procedure, the locations in the body of the catheter and the imaging instrument are known. Thus, the point-of-interest is projectable and displayable in the image even in cases in which the body moves relative to the imaging instrument. The system of this patent, however, does not determine whether the point of interest is in need of a medical treatment, not does it provide a medical treatment.

WO 02/38064 discloses a catheter apparatus and method for treating a plurality of adjacent locations of a tissue along a line of treatment, so as to form a continuous line of treatment in the tissue. The catheter apparatus includes (a) a catheter having an active site for applying the treatments at the adjacent locations along the line of treatment; (b) a positioning mechanism for sequentially guiding a single active site of the catheter along, or for positioning a plurality of active sites, along the line of treatment; c) an activating mechanism for activating the active site or sites to apply the treatment to the tissue at the adjacent locations; and (d) at least one position sensing mechanism for determining positions in the tissue at which the treatments have been applied. The system of this publication, however, does not determine whether the tissue is need of a treatment or which treatment is needed.

### SUMMARY OF THE INVENTION

The present invention provides a system and method for treating body tissue. The system of the invention includes a device containing a probe unit. The probe unit includes one or more treatment probes and one or more tissue monitoring probes. The probe unit also includes one or more tissue sensing/characterization probes, for characterizing a tissue site.

During delivery of a treatment to a treatment site, e.g. a tissue site, from the one or more treatment probes, the monitoring probes obtain, essentially in real-time, measurements of one or more parameters of a the state of the tissue site being treated. A processor is configured to receive signals from the one or more monitoring probes indicative of the measurements taken at the tissue site being treated. The processor is further configured to activate the treatment probes and the monitoring probes so as to deliver to the tissue site the determined treatments while receiving, in real-time, instantaneous signals indicative of a state of the treatment site during treatment progression from the tissue monitoring probes. The treatment monitoring signals are used by the processor to determine the optimal manner of treatment progression, modifying the treatment as necessary, in order to achieve the optimal treatment plan.

A treatment probe may be, for example, a treatment probe that performs, a physical treatment such as ablation, cryosurgery, microsurgery or taking a biopsy, a chemical treatment such as application of a toxin or drug to the tissue site, a biological treatment, such as DNA therapy, viral therapy and enzyme therapy.

The measurements obtained by the monitoring probes may include a physical measurement, such as density or fluidity, a chemical measurement, such as acidity, or a biological measurement, such as an enzymatic activity, or a level of gene expression activity. The processor may, on the basis of the sensing measurements, classify the tissue at the tissue site. The tissue classification may relate to the presence or absence of a malignancy or pre-cancerous state or to the presence or absence of a pathology such as internal bleeding. The processor then determines, on the basis of the classification, one or more treatments to be delivered to the tissue site. The processor is further configured to activate the treatment probes and the tissue monitoring probes so as to deliver to the tissue site the determined treatments while receiving, in real-time, instantaneous signals indicative of a state of the tissue site during treatment progression from the tissue monitoring probes. The treatment monitoring signals are used by the processor to determine a manner of treatment progression, modifying the treatment as necessary.

The method of the invention involves delivering the probe unit to a tissue site to be treated. During treatment of the tissue site, signals are obtained from the monitoring probes and input to the processor in order to determine, in real time as the treatment is being carried out, whether the treatment carried out so far is adequate or whether an additional treatment needs to be carried out or whether the parameters of the treatment need to be modified.

The system and method of the invention may be used for treating tissue sites located on the skin surface or just below the skin surface. The system and method of the invention may be used for minimally invasive surgery, for example, by insertion either percutaneously or via a trocar valve. The tissue site to be treated may be located in a body lumen, or adjacent to a body lumen.

Thus, in its first aspect, the invention provides a system for analysis and treatment of a tissue site comprising:
(a) a device having a probe unit comprising
   i. one or more tissue sensing and monitoring probes configured to measure one or more parameters indicative of one or more states of the tissue site; and
   ii. one or more tissue treatment probes configured to deliver a treatment to the tissue site;
(b) a controller comprising a processor configured to:
   i. receive and process signals from the one or more tissue sensing and monitoring probes and determine whether the probe unit is located at the tissue site to be treated;
   ii. activate the one or more of the treatment probes and the one or more sensing and monitoring probes to monitor one or more states of the tissue site while delivering the one or more treatments to the tissue site; and
   iii. as the treatment proceeds, receive and analyze signals from the one or more tissue sensing and monitoring probes, indicative of one or more measurements obtained by the one or more tissue sensing and monitoring probes, determine whether the monitored tissue parameters correspond to the required treatment and determine whether the treatment parameters need to be modified or whether predetermined treatment targets have been achieved.

In its second aspect, the invention provides a device for analysis and treatment of a tissue site comprising:
(a) a probe unit comprising
   i. one or more tissue sensing and monitoring probes configured to measure one or more parameters indicative of one or more states of the tissue site; and
   ii. one or more tissue treatment probes configured to deliver a treatment to the tissue site.

In its third aspect, the invention provides a method for analysis and treatment of a tissue site comprising:
(a) delivering to the tissue site a probe unit comprising one or more treatment probes, one or more tissue sensing and monitoring probes;
(b) measuring one or more values of one or more parameters indicative of one or more states of said tissue site for determining whether the probe unit is located at a tissue site requiring treatment prior to activating the one or more treatment probes.
(c) activating the one or more treatment probes to deliver the one or more determined treatments to the tissue site;
(d) receiving signals from the one or more tissue sensing and monitoring probes during delivery of the treatment indicative of one or more measurements obtained by the one or more tissue monitoring probes; and
(e) determining whether the monitored tissue parameters correspond to the required treatment and determine whether the treatment parameters need to be modified or whether predetermined treatment targets have been achieved.

### BRIEF DESCRIPTION OF THE DRAWINGS

In order to understand the invention and to see how it may be carried out in practice, embodiments will now be described, by way of non-limiting example only, with reference to the accompanying drawings, in which:

FIG. 1 shows a system for analysis and treatment of body tissue, in accordance with one embodiment of the system of the invention;

FIG. 2 shows exemplary arrangements of the probe unit;

FIG. 3 shows a system for analysis and treatment of body tissue, in accordance with another embodiment of the system of the invention;

FIG. 4 is a flowchart of a method for tissue analysis and treatment in accordance with one embodiment of the method of the invention;

FIG. 5 is a flowchart of a method for tissue analysis and treatment in accordance with another embodiment of the method of the invention;

FIG. 6 shows the method of the invention applied to analysis and treatment of a tissue site in a body lumen;

FIG. 7 shows the method of the invention applied to analysis and treatment of tissue site located adjacent to a body lumen;

FIG. 8 shows the method of the invention applied to analysis and treatment of a tissue site located on a skin surface;

FIG. 9 is a schematic illustration of an example of a system for in-situ treatment of body tissue, configured and operable according to an example of the present invention;

FIG. 10 is a flowchart of an example of a method for in-situ treatment of body tissue, in accordance with an example of the present invention;

FIG. 11 is a flowchart of another example of a method for in-situ treatment of body abnormalities, in accordance with an example of the present invention;

FIGS. 12A-12C are schematic illustrations of different examples of probes suitable to be used in the system of the present invention;

FIG. 13 is a schematic illustration of another configuration of a system for in-situ treatment of body tissue, in accordance with an example of the present invention; and

FIG. 14 is a flowchart of a method for in-situ characterization and treatment of a lesion, in accordance with an example of the present invention.

### DETAILED DESCRIPTION OF EXEMPLARY EMBODIMENTS

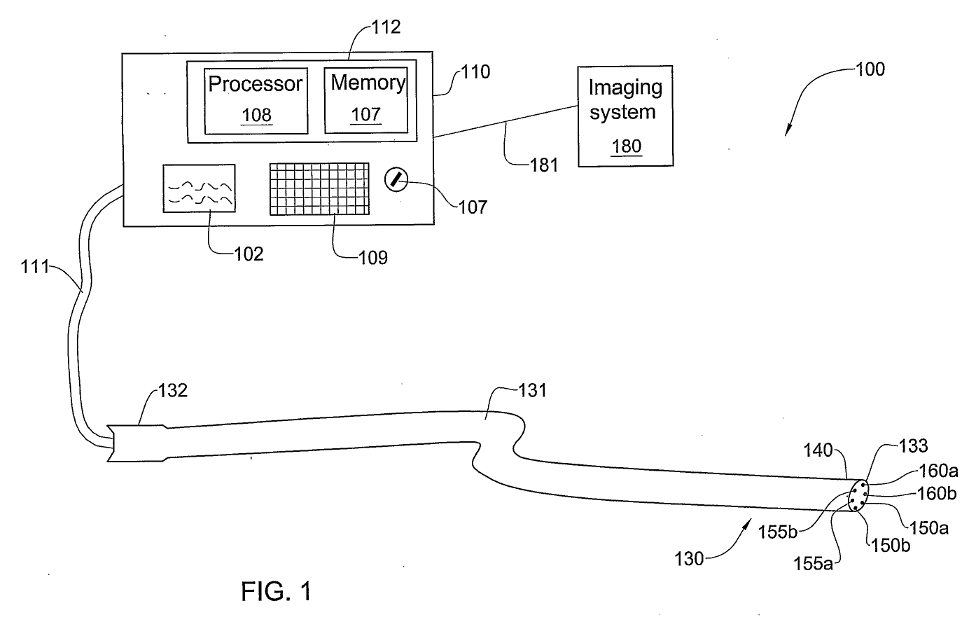

FIG. 1 shows a tissue analysis and treatment system 100 in accordance with one embodiment of this aspect of the present invention. The system 100 includes a probe device 130 having a probe unit 140. In the system 100 shown in FIG. 1, the probe device 130 is a catheter having a slender shaft 131 that may be flexible or rigid. The probe device 130 has a proximal end 132 and a distal end 133, also referred to herein as the catheter “tip”. Using a probe device 130 in the form of a catheter is useful when the tissue to be treated is located inside the body. This is by way of example only, and the probe device may have any configuration as required in any application.

According to some embodiments of the present invention the probe device 130 may be implemented using the embodiments below:

In accordance with an embodiment of the present invention, the probe device 130 may be an extracorporeal, hand-held, and may include a handle, for easy carrying.

In accordance with another embodiment of the present invention, the probe device 130 may be employed for minimally invasive surgery, for example, for insertion via a trocar valve, or for another percutaneous insertion.

In accordance with yet another embodiment of the present invention, the probe device 130 may be an intracorporeal probe, adapted for insertion via body orifice to a body lumen, for characterizing a portion of inner lumen wall.

In accordance with still another embodiment of the present invention, the probe 130 device may be an intracorporeal probe, adapted for percutaneous insertion to a body lumen, for characterizing a portion of inner lumen wall.

In accordance with yet another embodiment of the present invention, the probe device 130 may be an intracorporeal probe, adapted for insertion via body orifice to a body lumen, and for penetrating the lumen, for characterizing a portion of subcutaneous tissue.

In accordance with still another embodiment of the present invention, the probe device 130 may be an intracorporeal probe, adapted for percutaneous insertion to a body lumen, and for penetrating the lumen, for characterizing a portion of subcutaneous tissue.

In accordance with yet another embodiment of the present invention, the probe device 130 may be an adapted for characterizing a portion of subcutaneous tissue

In accordance with yet another embodiment of the present invention, the probe device 130 may be an adapted for characterizing a portion of subcutaneous tissue, in open surgery.

In accordance with yet another embodiment of the present invention, the probe device 130 may be extracorporeal, adapted for characterizing a portion of skin. Other embodiments may have other configurations and capabilities.

The probe unit 140 communicates with a control unit 110. For example, communication between the probe unit 140 and the control unit 110 may be via a wired connection. In this case, as shown in FIG. 1, the proximal end 132 of the probe device 130 is connected to the control unit 110, via a cable 111. Alternatively, the probe unit 140 and the control unit 110 may communicate via a wireless connection. The control unit 110 includes a computer processing unit (CPU) 112 having a processor 108 and a memory 107. A data input device such as a keypad 109 or a joystick 107 is used to input data to the CPU 112. A monitor screen 102 displays relevant information to the operator, as described below.

In the embodiment of FIG. 1, the probe unit 140 is located at the tip of the probe device 130. The probe unit 140 is an integrated unit that includes one or more treatment probes 160 for treating a tissue site together with one or more monitoring probes 155. Two monitoring probes 155a, 155b and two treatment probes 160a, 160b are shown in FIG. 1. This is by way of example only, and the system 100 may have any number of monitoring probes 155 and treatment probes 160 as required in any application. The probe unit 140 may optionally include one or more tissue characterizing or sensing probes 150 for characterizing a tissue site.

The one or more treatment probes 160 deliver one or more treatments to the tissue site. A treatment probe 160 may be, for example, an injector, or a bombardment device. A treatment probe 160 may also be a device for localized surgery such as a device for resection, cryosurgery, or laser surgery, or a device for ablation, such as an ultrasound, RF, MW ablation device. A treatment probe 160 may also be a dispensing instrument, for example, for dispensing a substance at a tissue site, such as a medication or brachytherapy seeds.

In accordance with the invention, the monitoring probes 155 are adapted to monitor a condition of the treated tissue site in real time during delivery of treatment by the treatment probes 160. The tissue monitoring probes may also function as tissue characterization probes, or the probe device 140 may have different monitoring probes 155 and tissue characterization probes 150, as shown in FIG. 1.

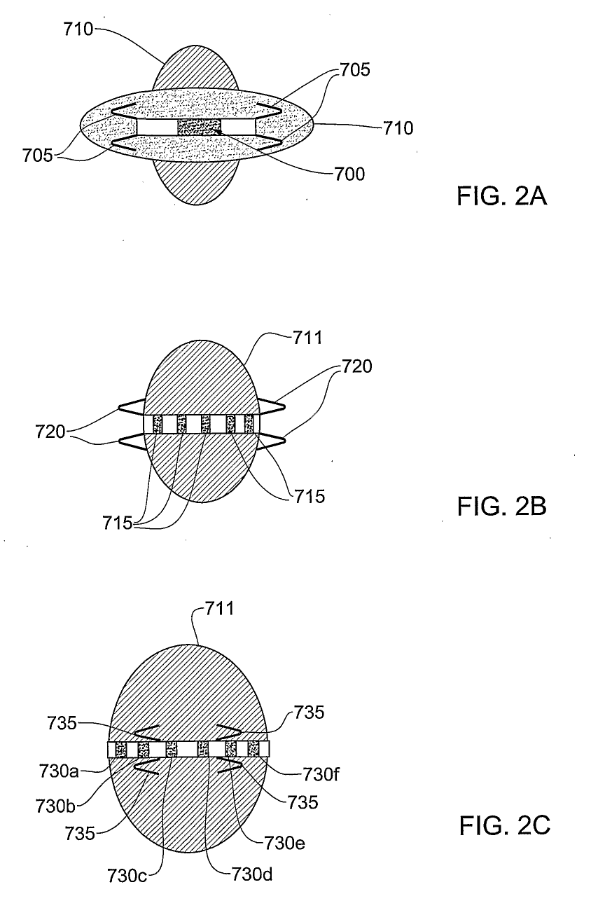

FIG. 2 shows exemplary arrangements of the probes in the probe unit 140. In FIG. 2a, a tissue monitoring probe 700 is located between a treatment probe 705 having several extensions, so that the monitoring probe 700 is positioned adjacent to the center of the tissue site 710 that receives the treatment or treatments by the treatment probe 705. The monitoring probe 700 monitors the tissue state in a region 711 in real time during delivery of the treatment to the treatment site 710, as explained below.

In FIG. 2b, a plurality of tissue monitoring probes 715 are located between a treatment probe 720. The monitoring probes 715 may all be of the same type, or some probes may be different from others. The monitoring probes 715 monitor the tissue state in a region 711 in real time during delivery of the treatment to the treatment site. Two or more of probes 715 may sense and/or monitor the entire monitoring site 711, or each probe 715 may sense and/or monitor a different portion of the monitoring site 711.

The probe unit arrangement shown in FIG. 2c has a plurality of monitoring probes 730. The probes 730c and 730d are located between the tissue treatment probe 735, while the probes 730a, 730b, 730e and 730f are located beyond the treatment probe 735. Some or all of the tissue monitoring probes 730 may also serve as tissue characterization probes. The monitoring probes 730 monitor the tissue state in a region 711 in real time during delivery of the treatment to the treatment site. Two or more of probes 730 may sense and/or monitor the entire monitoring site 711, or each probe 730 may sense and/or monitor a different portion of the monitoring site 711.

A monitoring probe 155 may be, for example, an optical sensor, an X-ray sensor, a radiofrequency (RF) sensor, a microwave (MW) sensor, an infrared thermography sensor, an ultrasound sensor, a magnetic resonance (MR) sensor, an impedance sensor, resistivity sensor, capacitance sensor, electric field sensor, magnetic sensor, radiation sensor, an acoustic sensor a thermistor or temperature sensor, a thermocouple, a biosensor, a chemical sensor, a radioactive-emission sensor, or a mechanical sensor.

A tissue monitoring probe 155 may also be, for example, as disclosed in International Published Application WO 2006/103665, entitled “Electromagnetic sensors for tissue characterization” and assigned to the common assignee of the present application, the contents of which are hereby incorporated herein in their entirety by reference. This published patent application discloses a tissue sensing probe including a resonating element, formed as a conductive structure, and configured to be placed at an edge of a tissue site without penetrating into the tissue. The probe has a diameter-equivalent D, which defines a cross-sectional area of the resonating element on a plane substantially parallel with the edge. The resonating element is configured to resonate with a free-air wavelength in the range of about λ-10λ, wherein λ is at least about ten times the diameter-equivalent D. Upon receiving a signal in the range of about λ-10λ, the sensor induces electric and magnetic fields in a “near zone” in the tissue, the near zone being hemispherical with a diameter of substantially D, while causing negligible radiation in a “far zone”, so that the tissue, in the near zone effectively functions as part of the resonating element, varying a resonating response to the sensor. The tissue, in the near zone is characterized by its electromagnetic properties by the resonating response of the sensor.

By way of another example, a tissue monitoring probe 155 may be a non-irradiative electromagnetic sensor for tissue characterization, for example, as taught in commonly owned U.S. Pat. No. 6,813,515 entitled “method and system for examining tissue according to the dielectric properties thereof”, whose disclosure is incorporated herein in its entirety by reference. This patent describes a non-irradiative electromagnetic sensor, which applies an electrical pulse to a tissue, thus generating an electrical fringe field in the zone of the tissue and producing a reflected pulse therefrom with negligible radiation penetrating into the tissue itself. The sensor detects the reflected electrical pulse and compares the electrical characteristics of the reflected electrical pulse with respect to the applied electrical pulse to provide an indication of the dielectric properties of the examined tissue.

The tissue characterization probes 150, if present, measure a value of one or more parameters of the tissue site in order to facilitate delivery of the probe unit 140 to the tissue site to be treated, and may be for example of any type mentioned above in reference to the tissue monitoring probes 155.

If a plurality of tissue characterization probes 150 or tissue monitoring probes 155 is employed, they may be arranged in the probe unit 140 in a curved array to provide three-dimensional information on the tissue site, for example, using small-scale computerized tomography.

The probe device 130 may be a cannula having one or more working channels configured to receive the treatment probes 160, the monitoring probes 155 and the tissue characterization probes (when present) required for the specific application. The various probes may be introduced into separate working channels and positionable simultaneously in the probe unit 140.

The probe unit 140 is preferably configured to be detectable by an external imaging system 180 such as an X-ray, ultrasound or MRI imaging system, in order to facilitate delivery of the probe unit 140 to a desired body site, and to locate the probe unit 140 in the body. The imaging system 180 may communicate with the CPU 112 over a communication channel 181 that may be wired or wireless. The device 130 may be, for example, a navigable catheter as disclosed in the above cited U.S. Pat. No. 6,947,788.

The CPU 112 may be configured to receive signals from one or more characterization probes 150 during delivery of the probe unit 140 to the tissue site to be treated indicative of the values of tissue parameters measured by the tissue characterization probes at the tissue site where the probe unit 140 is located. The CPU 112 analyzes the signals received from the characterization probes 150 and determines whether or not the probe unit 140 is positioned at a tissue site having one or more properties of the site to be treated.

When the probe unit 140 is located at the tissue site to be treated, one or more treatments are delivered to the tissue site by the treatment probes 160. During delivery of the treatment, the monitoring probes 155 measure in real time values of parameters indicative of the state of the tissue. Signals from the monitoring probes 155 indicative of the state of the tissue being treated are analyzed by the CPU 112, in order to monitor progress of the treatment, to modify the form of the treatment, to adjust the treatment, or the treatment plan.

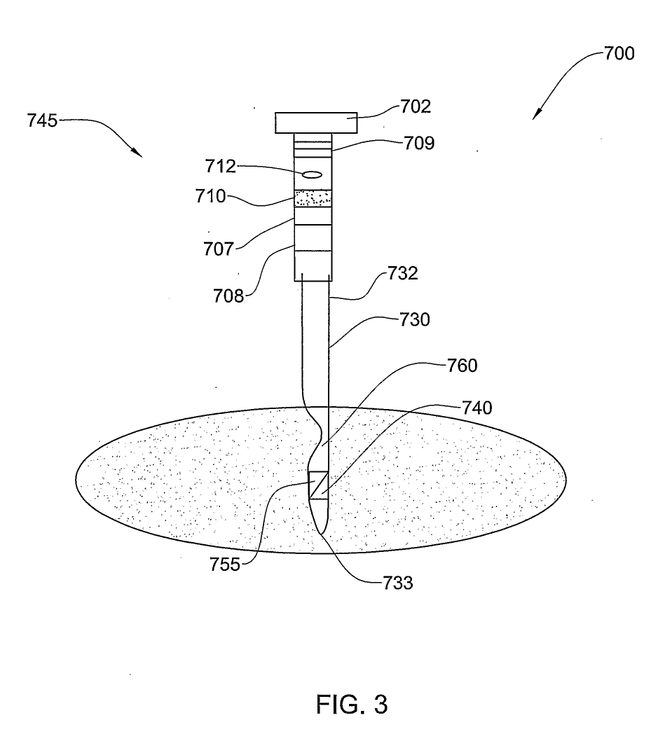

FIG. 3 shows a tissue analysis and treatment system 700 in accordance with another embodiment of this aspect of the present invention. The system 700 is particularly useful when the treatment to be delivered is a tissue biopsy and the tissue site to be treated is located near the body surface. The system 700 includes a probe unit 740 located at the distal end, or tip 733, of a rigid cannula 730. The probe unit 740 includes one or more monitoring probes 755. The system 700 includes as a treatment probe e.g. a biopsy tool 760. The proximal end 732 of the cannula 730 is attached to a handle 745 which houses a control unit 710. The control unit 710 includes a computer processing unit (CPU) having a processor 708 and a memory 707. A data input device such as a keypad 709 is used to input data to the CPU 112. A monitor screen 702 or one or more LEDs 712 may be used to display relevant information to the operator. The cannula tip 733 may be pointed, as shown in FIG. 3 in order to facilitated insertion when the cannula tip 733 is to be inserted into the body percutaneously.

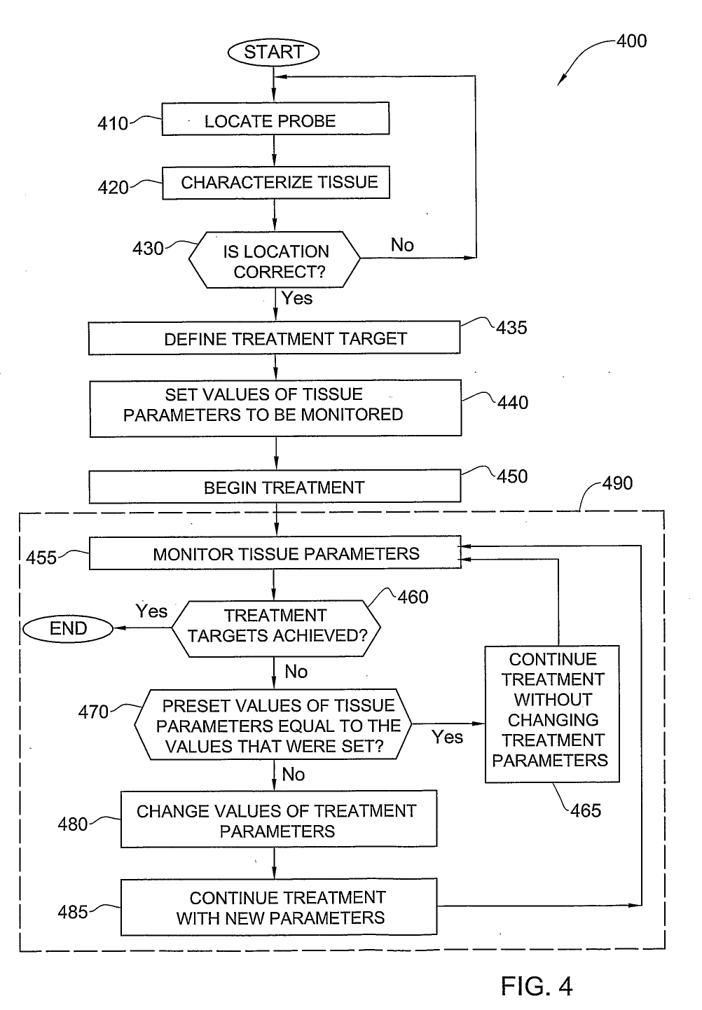

Reference is now made to FIG. 4, exemplifying a flowchart 400 of a method for tissue analysis and treatment in accordance with one embodiment of this aspect of the invention. In a locating step 410, the probe 130 is placed, for example inside a lumen or on the surface of part of the body and is oriented proximal to or touching some tissue. In a characterizing step 420, at least one sample of the tissue is characterized to determine the cell type and tissue type. For example, it is determined at this stage, whether some or all of the tissue is abnormal tissue, such as cancerous tissue. Preferably, the processing unit characterizes the tissue proximal to the sensor probe and displays results on the monitor 102. In some other embodiments, the results are produced graphically, numerically, or as positive or negative answers. The results may also be presented textually.

The tissue characterization relating to the abnormal tissue may relate to the detection of a malignancy, or a pre-cancerous state. Additionally or alternatively it may relate to the detection of another pathology, for example, internal bleeding.

In a checking step 430, it is determined whether the location of the probe is correct. If negative, the probe is moved to another location. The relocation of the probe may be manual, semi-manual or automatic employing for example, a two-dimensional or three-dimensional computer controlled stage, as is known in the art. There may be a computer program which controls the stage and defines the sequence of moving the probe from one location to the next. In cases, where the relocation is manual or semi-manual, the system may provide the operator with specific instructions on how and to whereto move the probe.

Additionally or alternatively, further steps of moving the stage may be introduced in response to the results of step 430. Once the probe 130 is relocated, steps 420-430 are repeated. If in step 430, the location of the probe is correct; a process to monitor a tissue site and treat the tissue is preformed.

Thereafter, in a defiling target step 435, the treatment process targets are defined. The treatment of defining target 435 may be selected from, but is not limited to, at least one physical treatment; at least one chemical treatment; at least one biological treatment or to mixtures thereof. Examples of physical treatment include, but are not limited to, ablation, cryosurgery and microsurgery. Examples of chemical treatment include, but are not limited to applying a toxin, or drug to the tissue. Toxins are exemplified, but not limited to neurotoxins, fungal toxins and bacterial toxins. Some examples of drugs which can be used in the method of this invention are chemotherapeutic agents, oxidizing agents and antibiotics, though any other drug known in the art or to be discovered in the future may possibly be used in the method and system of the present invention. Examples of biological treatment include, but are not limited to DNA therapy, viral therapy and enzyme therapy. The treatment may comprise a substance to be applied to the tissue e.g. a nucleic acid encoding, for example, a growth factor, a protein, such as a growth factor, and a cell expressing a protein such as a growth factor, so as to effect gene therapy, revascularization, such as myocardial revascularization, or to accelerate/decelerate cell growth and/or differentiation. The treatment may also comprise a combination treatment such as chemotherapy and laser ablation. Whatever the type of treatment, it may be applied locally employing the system of the present invention.

Following defining target step 435, in step 440 the values of the one or more tissue parameters that need to be monitored by the tissue monitoring probes 155 are set by the CPU 112. In step 450 treatment delivery begins, and the process enters a control cycle 490.

In step 455 the tissue parameters are continuously and in real-time monitored by tissue monitoring probes 155. During the delivery of treatment in step 450, the CPU determines in step 460, essentially in real-time, on the basis of measurements obtained by the one or more monitoring probes 155, whether the treatment targets which were defined in step 435 were achieved or whether additional treatment needs to be carried out (with or without changing the value of the treatment parameters). If yes, the treatment process is complete and the process terminates. If no, then the monitoring and treatment process enters a control cycle 490, which includes the following steps: in step 470 it is determined in real-time during delivery of the treatment, whether the present values of the tissue parameters are equal to the values that were preset in step 440. If yes, then in step 465 the treatment process is continued (and monitored in real time in step 455) and the process returns to step 455. If no, in step 480 the values of the treatment parameters which were set in step 400 are changed, the treatment continues with the new treatment parameters, and the process returns to step 455 as the monitoring process is continued continuously in real time in the a control cycle 490

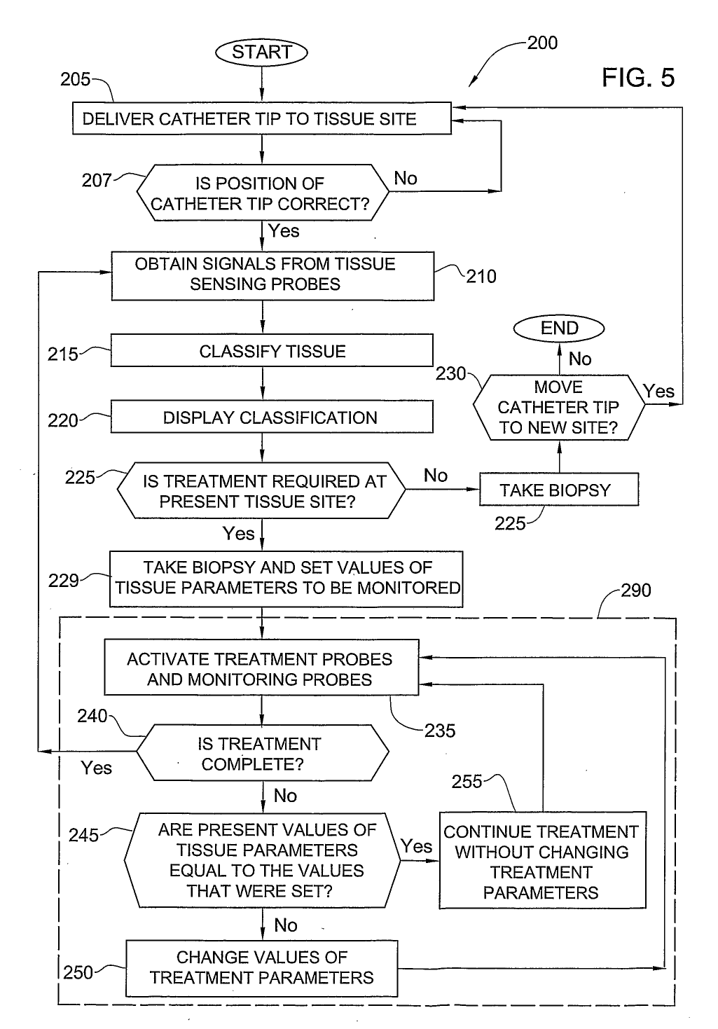

FIG. 5 shows a flowchart 200 for a method for tissue analysis and treatment in accordance with another embodiment of this aspect of the invention. In step 205, the probe unit 140 is delivered to a tissue site to be treated. The tissue site to be treated may be located on the body surface, or may be an internal body site. If the site to be treated is internal, the catheter tip maybe inserted into the body through a body office, such as the mouth, nostril, anus and so on. Alternatively, the catheter tip may be introduced percutaneously through an incision in the skin. Movement of the probe unit 140 may be manual, semi-manual or automatic employing for example, a two-dimensional or three-dimensional computer controlled stage (not shown). The probe unit 140 may be imaged by the imaging system 180 in order to assist the operator in delivering the probe unit 140 to the tissue site to be treated. In cases where the movement is manual or semi-manual, the system may provide the operator with specific instructions on how to manipulate the device 130 in order to deliver the probe unit 140 to a desired location.

In step 207 it is determined whether the probe unit 140 is located at the desired tissue site. If no, then the process returns to step 205 with the probe device being moved to a new tissue site. If the probe device is located at the desired tissue site, then in step 210, signals obtained from the one or more tissue sensing probes indicative of one or more measurements obtained by the tissue sensing probes at the tissue site are input to the CPU 112. The measurements may include a physical measurement, such as density or fluidity, a chemical measurement, such as acidity, or a biological measurement, such as an enzymatic activity, or a level of gene expression activity. In step 215 the CPU 112 analyzes the signal or signals from the tissue sensing probes and classifies the tissue at the tissue site. The tissue classification may relate to the detection of a malignancy, or a pre-cancerous state. Additionally or alternatively it may relate to the detection of a pathology such as internal bleeding. The classification may be relative, that is, a comparison of the results obtained by the sensing probes to data previously obtained on one or more reference tissues and stored in the memory 107. The classification generated by the CPU 112 may be displayed on the monitor 102 (step 220). The results may be displayed graphically, numerically or textually.

In step 225, it is determined, preferably in real-time, based upon the classification of the tissue at the present tissue site, whether a treatment is required at the treatment site. If no treatment is required at the present treatment site, then in step 227 a biopsy may be taken and in step 230 it is determined whether the probe unit 140 is to be moved to a new tissue site. If yes, then the process returns to step 205 with the probe unit 140 being moved to a new tissue site. If at step 230 it is determined that the probe unit 140 is not to be moved to a new tissue site then the process ends. If at step 225 it is determined that a treatment is needed at the present tissue site, then in step 229 the values of the tissue parameters to be monitored are initiated and a biopsy may be taken. The monitoring and treatment process then enters a control cycle 290, which includes the following steps. In step 235 the CPU activates one or more of the treatment probes 160 and one or more of the monitoring probes 155 in order to effectuate the required treatment and to monitor the state of the tissue site in real-time during delivery of the treatment. The operator may first input to the CPU 112 using the keypad 109 the parameters of the treatment. The parameters of the treatment may be proportional, inversely proportional, a derivative of, an integral of, or some other relation to results of the tissue classification step. In some cases, the level of treatment is varied over a continuous range, in other cases parameters of the treatment can only take on discrete values.

The treatment carried out in treatment step 235 may be one or more physical treatments; one or more chemical treatments one or more biological treatments or a combination thereof. Examples of physical treatments include ablation, cryosurgery and microsurgery. Examples of chemical treatment include application of a toxin or drug to the tissue site. The applied substance may become activated after having been taken up by the tissue, for example, as in photodynamic therapy. Toxins are exemplified by fungal toxins and bacterial toxins. Some examples of drugs which can be used in the method of this invention are chemotherapeutic agents, oxidizing agents and antibiotics, though any other drug known in the art or to be discovered in the future may possibly be used in the method and system of the invention. Examples of biological treatments include, but are not limited to, DNA therapy, viral therapy and enzyme therapy. The treatment may comprise a substance to be applied to the tissue e.g. a nucleic acid encoding, for example, a growth factor, a protein, such as a growth factor, and a cell expressing a protein such as a growth factor, so as to effect gene therapy, revascularization, such as myocardial revascularization, or to accelerate/decelerate cell growth and/or differentiation. The treatment may also comprise a combination treatment such as chemotherapy and laser ablation. Whatever the type of treatment, it may be applied locally employing the system of the present invention.

During the delivery of treatment in step 235, the CPU determines in step 240, essentially in real-time, on the basis of measurements obtained by the one or more monitoring probes 155, whether the treatment that has been carried out so far at the present tissue site is adequate or whether additional treatment needs to be carried out. If no, in step 245 it is determined in real-time during delivery of the treatment, whether the present values of the tissue parameters are equal to the values that were preset in step 229. If yes, then in step 255 the treatment process is continued and the process returns to step 235 where the treatment is monitored in real time If no, in step 250 the values of the treatment parameters which were set in step 229 are changed and the monitor process is continued continuously in real time in the control cycle 290.

If at step 240 it is determined that the treatment is complete, then the process may return to step 210, if it is desired to classify the tissue site again following the treatment as shown in FIG. 5. Alternatively, if at step 240 it is determined that the treatment is complete e.g. the treatment targets which were defined in step 235 were achieved, the process may return directly to step 225, 227 or 230. If further treatment needs to be performed at a tissue site, the type of treatment and/or the dosage of the treatment may change each time or may remain the same.

The CPU 112 may be configured to generate a status mapping of the tissue sites that have been treated and to display the map on the monitor 103. The map is updated after the completion of the treatment at each tissue site to be treated. Thus, the operator can see whether all areas of the tissue have been treated and to what degree they have been treated.

Some specific embodiments of the present invention are directed to treating respiratory tract abnormalities. For example, the invention may be directed to the treatment of a bronchial carcinoid, formerly known as a bronchial adenoma. The carcinoid may be benign or malignant, typically having a prolonged course. In some cases, an endobronchial portion of the tumor may obstruct the lumen of one or more major bronchi. Other embodiments of the invention are used to treat metastases of the respiratory tract which result from primary cancers of the body, such as from the breast, prostate, colon, kidney, bone and the like or from melanoma.

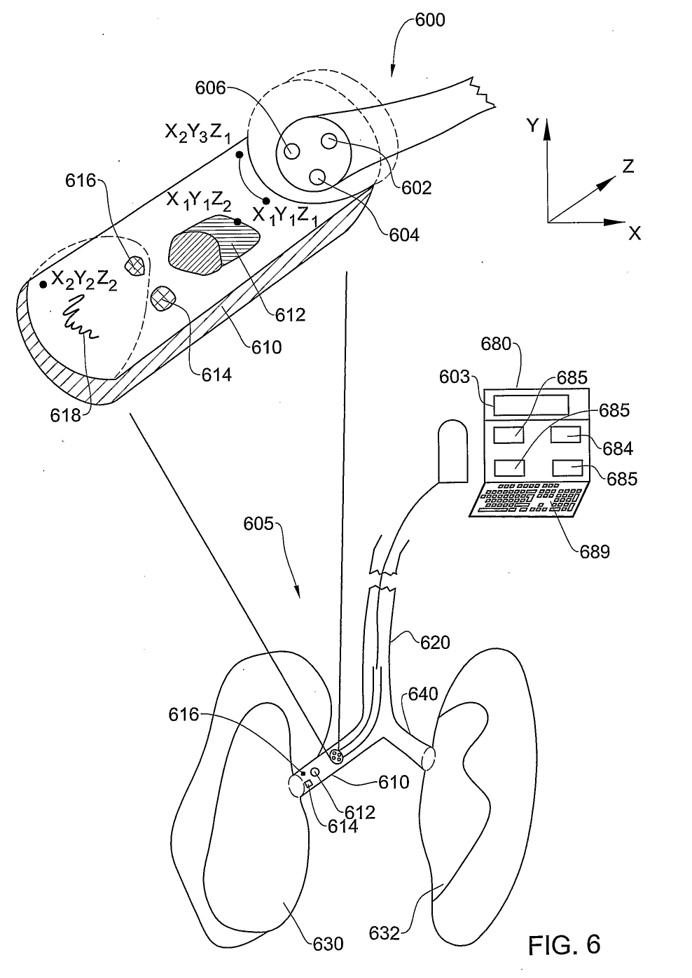

Reference is now made to FIG. 6 which shows an example of use of the method of the invention for treatment of tissue site in a body lumen. In the example of FIG. 6, the tissue site is a three-dimensional respiratory tract abnormality. Shown in FIG. 6, are a portion of a respiratory tract 605 including a trachea 620 connected a right bronchus 610, a left bronchus 640, a left lung 630 and a right lung 632. Abnormal tissue located at tissue sites 612, 614 and 616 in the lumen of the right bronchus 610 is to be treated by laser ablation using the system 100 of FIG. 1. The probe unit 140 is delivered to a first tissue site. The tissue at the tissue site at the first location is sensed by the one or more tissue sensing probes 150. The tissue sensing probes send signals to the CPU 112. The signals are processed by the CPU 112 which provides an output to the monitor screen 102 indicating that the tissue at the first tissue site is normal. The probe unit 140 is now moved to a second tissue site. The tissue at the second tissue site is analyzed and the tissue is found to be normal. No treatment is delivered at sites of normal tissue.

The probe unit 140 is now moved to a third tissue site which is sensed by the one or more tissue sensing probes 150 that send signals to the CPU 112. The signals are analyzed by the CPU 112 and results of the analysis are displayed on the monitor 109 indicating that the tissue at the third tissue site is abnormal. The duration, quantity and quality of the laser ablation treatment may be determined at this stage.

The CPU 112 activates a laser, such as a Yag-Niobium laser. The laser beam is conducted by an optic fiber along the cable 111 and the device 130 to the probe unit 140 where the laser light energy is delivered to the third tissue site. The tissue sensing probes then sense the treated tissue at the third tissue site, and sends signals to the CPU 112. In accordance with the displayed output, the operator can determine, what, if any, further treatment is required at the third tissue site. Alternatively, the determination may be made by the CPU 112, based on a set of predetermined rules.

If the treatment at the third location is complete, the apparatus is moved to a fourth position. The process is repeated until all of the abnormal tissue at the tissue sites 612, 614, 616 and 618 has been treated. Thereafter, the system may perform a full scan at all locations to verify that all the abnormal tissue has been treated.

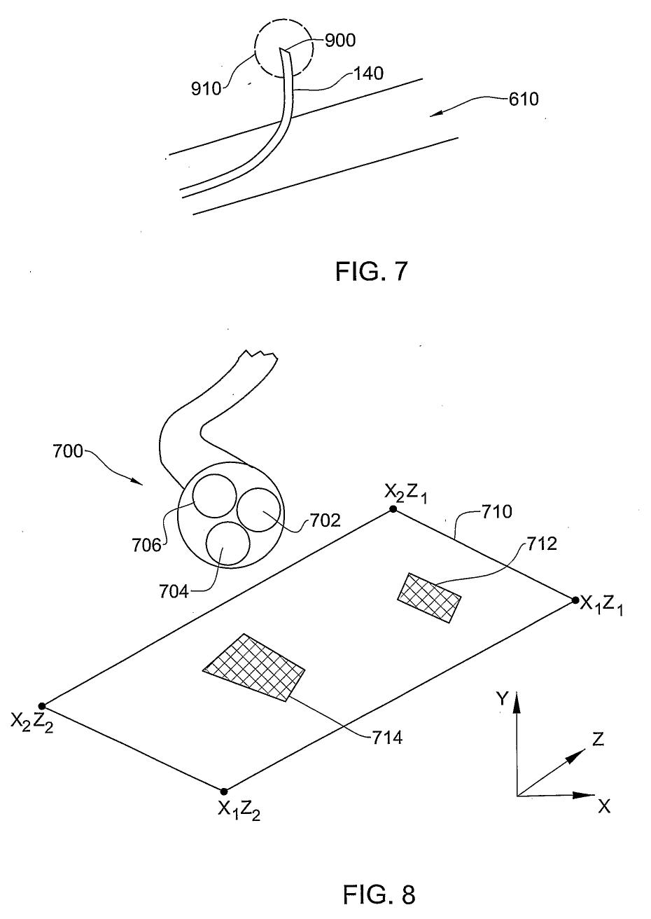

The probe device 130 may be adapted to leave a body lumen and enter into an adjacent body region. For example, as shown in FIG. 7, the probe device 140 may have a pointed tip 900 to allow the tip to pass through the wall of a lumen, such as a bronchus 610 in order to arrive at a tissue site 910 to be treated.

Reference is now made to FIG. 8, which is a schematic illustration of an example of a method for treatment of a substantially two-dimensional abnormality on a skin surface, in accordance with the invention. The probe unit 140 of the system 100 is moved over a skin surface area in which there are sites 712, 714 of abnormal tissue which are to be treated by the method of FIG. 4 or 5.

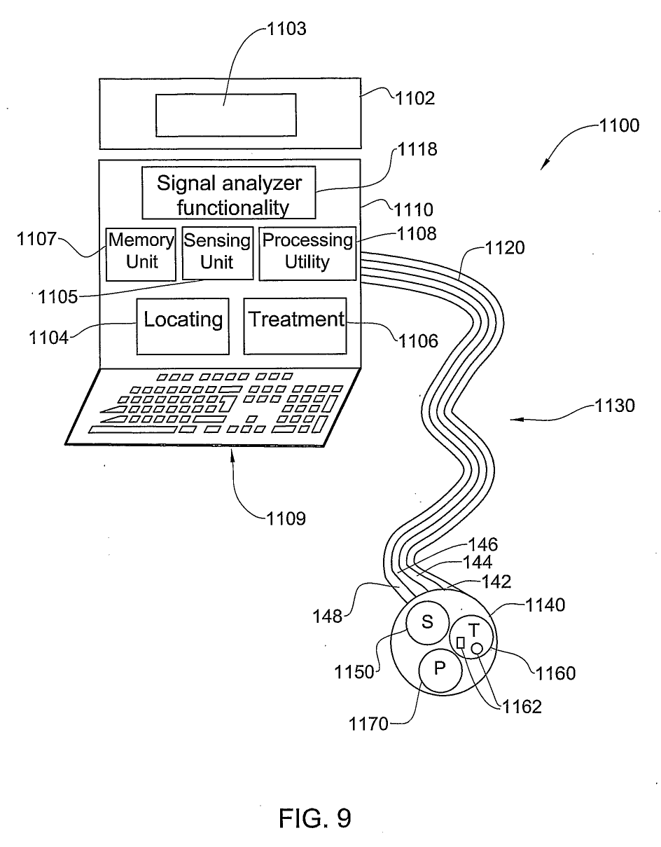

Reference is now made to FIG. 9, which is a schematic illustration of an example of a treatment managing system 1100 of the present invention. System 1100 is designed for in-situ treatment of a tissue at a plurality of locations of a body, so as to form a continuous or segmented area of treatment of the tissue.

System 1100 includes an apparatus 130 configured for proximal orientation to the tissue in the body. Apparatus 1130 includes an active head 1140 including a sensing probe 1150 for characterizing a sample of tissue from at least one of the proximal locations. Such a sensor may include an optical sensor, an X-ray sensor, an RF sensor, a MW sensor, an infrared thermography sensor, or an ultrasound sensor, an MR sensor, an impedance sensor, a temperature sensor, a biosensor, a chemical sensor, a radioactive-emission sensor, and a mechanical sensor.

System 1100 is configured to be connectable to one or more external units, generally at 1110. The latter is typically a computer system including inter alia a memory utility 1107, a data processing and analyzing utility 1108 associated with a signal analyzer functionality 1118 and a data input utility (keyboard) 1109, and a display unit 1102. Further provided in system 1100 are a locating utility 1104, and at least one treatment unit 1106.

Head 1140 preferably includes a positioning mechanism 1170 to enable sequential guiding of the apparatus from at least one of the plurality of locations to one or more other locations of the plurality of locations. Apparatus 1130 further includes a treatment probe 1160. The treatment probe has at least one active outlet 1162 for applying at least one treatment to at least one of the plurality of proximal locations for treating the sample. Probe 1160 includes an activating mechanism (not shown) adapted to activate the at least one outlet.

Apparatus 1130 and/or treatment probe 1160 may include an instrument for localized surgery, for example, by resection, ablation, for example, of ultrasound, RF, MW or another ablation method, or by cryosurgery, laser surgery, and the like, a dispensing instrument, for example, for dispensing a medication or for implanting brachytherapy seeds, or an instrument for other characterization and/or treatment procedures.

Sensing probe 1150 may be of any known suitable type. In some embodiments of the invention, the sensing probe is configured as disclosed in commonly owned U.S. Patent Application 60/665,842.

Such sensing probe includes a resonating element, formed as a conductive structure, configured to be placed proximally to an edge of a tissue for characterization, without penetrating the tissue; and at least one conductive lead, for providing communication with an external system. The probe has a diameter-equivalent D, which defines a cross-sectional area of the resonating element, on a plane substantially parallel with the edge. The resonating element is configured to resonate at a free-air wavelength range of about λ-10λ, wherein λ is at least about ten times the diameter-equivalent D. Upon receiving a signal in the range of about λ-10λ, the sensor induces electric and magnetic fields, in a near zone, in the tissue, the near zone being a hemisphere having a diameter of substantially D, beginning with the edge, while causing negligible radiation in a far zone, so that the tissue, in the near zone, effectively functions as part of the resonating element, varying a resonating response to the sensor. The tissue, in the near zone, is characterized by its electromagnetic properties, by the resonating response to the sensor.

The sensor probe may be a nonirradiative electromagnetic sensor for tissue characterization, for example, as taught in commonly owned U.S. Pat. No. 6,813,515, whose disclosure is incorporated herein by reference with respect to this specific example. U.S. Pat. No. 6,813,515 describes a nonirradiative electromagnetic sensor, which applies an electrical pulse to a tissue, thus generating an electrical fringe field in the zone of the tissue and producing a reflected pulse therefrom with negligible radiation penetrating into the tissue itself. The sensor detects the reflected electrical pulse and compares the electrical characteristics of the reflected electrical pulse with respect to the applied electrical pulse to provide an indication of the dielectric properties of the examined tissue.

In some other embodiments of the invention, apparatus 1130 includes at least in part an endoscope tool. Such endoscope may be configured as a multi-channel endoscope or cannula, for carrying several instruments, for example, an optical instrument, a sensing probe, and another instrument such as a surgical instrument, which may be operated together. Alternatively, only one or two channels may be available, and instruments are pulled out and replaced with other instruments, as is required.

The sensor probe is preferably visible on other imaging modalities such as x-rays, ultrasound and MRI, and may be guided using another imaging modality, so that it can be guided to zones which are not accessible to an optical instrument or in cases where the optical instrument is not used.

In some cases, system 1100 is configured to measure reflection of electromagnetic fields from the near vicinity of the sensor probe, for example, as taught in commonly owned U.S. Pat. No. 6,813,515, or in commonly owned U.S. Patent Application 60/665,842. It will be appreciated that in accordance with embodiments of the present invention, other electromagnetic sensors may also be used.

Sensor probe 1150 may include a probe as disclosed in U.S. Ser. No. 11/196,732, filed on Aug. 4, 2005, entitled “Tissue-Characterization Probe With Effective Sensor-To-Tissue Contact”, assigned to the assignee of the present application.

Such probe is configured for tissue-characterization, being designed for effective sensor-to-tissue contact. The device includes an element, having a rigid surface of a linear cross-section, on which at least one sensor is arranged, and a mechanism for applying a force to a soft tissue, the line of force being at a sharp angle with the rigid surface, for stretching or stretching and pushing the soft tissue against the rigid surface, thus achieving effective contact between the tissue and the at least one sensor. In consequence, the accuracy of the sensing is improved. In accordance with another embodiment, a plurality of sensors is employed, arranged along a curved element, for providing three-dimensional information regarding the tissue, for example, by small-scale computerized tomography.

It will be appreciated that in accordance with embodiments of the present invention, other electromagnetic sensors may be used.

As indicated above, apparatus 1130 is associated with the external unit, being connected thereto by wires 1120. The external unit includes a sensing functionality 1105 adapted to receive at least one signal from sensing probe 1150 at each location and to analyze the at least one signal so as to provide at least one output. In some cases, functionality 1105 is part of processor 1108. Treatment functionality 1106 is adapted to receive the at least one output and provide at least one treatment to treatment probe 1160 responsive to the output. In some embodiments, the at least one treatment is provided by the treatment probe at one or more outlet 1162. In some cases, the treatment is only provided when the activating mechanism is activated. The activating mechanism is typically activated by a signal from the external unit.

In some embodiments, internal optical guides (of the kind known in the art) may be employed, such as a visual bronchoscope as part of apparatus 1130, connected to head 1140.

Both an external imaging mechanism, being part of locating apparatus 1104, and an internal optical guide may be used for guiding the head 1140 to the required location.

In one embodiment, a position sensing mechanism (locating apparatus 1104 of FIG. 9), is employed to determine the location of at least one of a plurality of locations for the treatment of the tissue, for example as is described in the above-indicated U.S. Pat. No. 6,711,429. Such mechanism is referred to therein as a locating sensor of a locating system. Such a locating system is understood to include an extracorporeal unit which defines a reference frame of coordinates and by interacting with the locating sensor serves to determine the position thereof in for example six degrees of freedom with respect thereto.

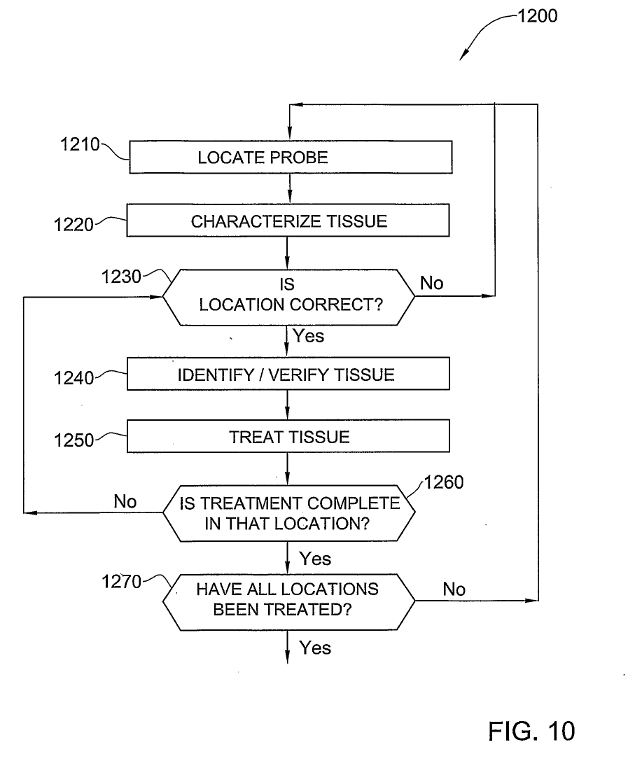

Reference is now made to FIG. 10, exemplifying a flowchart 1200 of a method for in-situ treatment of body tissue, in accordance with the present invention.

In a locating step 1210, the apparatus is placed inside a lumen or on the surface of part of the body and is oriented proximal to or touching some tissue. The apparatus used in this method may include, for example, the navigable catheter as disclosed in the above-indicated U.S. Pat. No. 6,947,788.

In a characterizing step 1220, at least one sample of the tissue is characterized to determine the cell type and tissue type. For example, it is determined at this stage, whether some or all of the tissue is abnormal tissue, such as cancerous tissue. In some embodiments, the method of the above-indicated U.S. Pat. No. 6,813,515 is used for examining tissue according to the dielectric properties thereof.

Preferably, the external unit analyzes the reflection proximal to the sensor probe and displays results on screen 1103. In some other embodiments, the results are produced graphically, numerically, or as positive or negative answers. The results may also be presented textually.

The results may be relative, that is, a comparison of results of abnormal tissue (such as tissue 612, 614, 616, 616 in FIG. 6 above relative to a reference tissue such as 610 of FIG. 6, or several references of the tissue taken from different locations. Alternatively, the results may be based on literary data, in which the tissue is characterized based on previous tests and/or data found in the literature.

The tissue characterization relating to the abnormal tissue may relate to the detection of a malignancy, or a pre-cancerous state. Additionally or alternatively it may relate to the detection of another pathology, for example, internal bleeding.

The user typically observes the results on a display of the tissue characterization step 220. In some cases, the user controls the level of treatment in response to the results of the tissue characterization step on a scale ranging from zero to 100%. In other cases, the treatment level scale is controlled automatically. The level of treatment may be proportional, inversely proportional, a derivative of, an integral of, or some other relation to results of the tissue characterization step. In some cases, the level of treatment is in a continuous range, in other cases it is performed in a stepwise manner.

In a checking step 1230, it is determined whether the location of the probe is correct. If negative, the probe is moved to another location. The relocation of the probe may be manual, semi-manual or automatic employing for example, a two-dimensional or three-dimensional computer controlled stage, as is known in the art. There may be a computer program which controls the stage and defines the sequence of moving the probe from one location to the next. In cases, where the relocation is manual or semi-manual, the system may provide the operator with specific instructions on how and to whereto move the probe.

Additionally or alternatively, further steps of moving the stage may be introduced in response to the results of step 1230. Once the probe is relocated, steps 1220-1230 are repeated. If in step 1230, the location of the probe is correct, a process to verify the tissue type is performed in verification step 1240.

Thereafter, in a tissue treating step 1250, the tissue is treated with a unit dosage of treatment. The unit is determined in response to the results of step 1240.

The treatment of treatment step 1250 may be selected from, but is not limited to, at least one physical treatment; at least one chemical treatment; at least one biological treatment or to mixtures thereof. Examples of physical treatment include, but are not limited to, ablation, cryosurgery and microsurgery. Examples of chemical treatment include, but are not limited to applying a toxin, or drug to the tissue. Toxins are exemplified, but not limited to neurotoxins, fungal toxins and bacterial toxins. Some examples of drugs which can be used in the method of this invention are chemotherapeutic agents, oxidizing agents and antibiotics, though any other drug known in the art or to be discovered in the future may possibly be used in the method and system of the present invention. Examples of biological treatment include, but are not limited to DNA therapy, viral therapy and enzyme therapy. The treatment may comprise a substance to be applied to the tissue e.g. a nucleic acid encoding, for example, a growth factor, a protein, such as a growth factor, and a cell expressing a protein such as a growth factor, so as to effect gene therapy, revascularization, such as myocardial revascularization, or to accelerate/decelerate cell growth and/or differentiation. The treatment may also comprise a combination treatment such as chemotherapy and laser ablation. Whatever the type of treatment, it may be applied locally employing the system of the present invention.

Following treatment step 1250, a checking step 1260 is performed to determine whether the treatment has been completed at that location. If affirmative, another checking step 1270 is performed to see whether the tissue has been treated in all locations. If the result of step 1260 is negative, then steps 1230-1260 are repeated. However, if the probe has been inadvertently moved, then this will be determined in step 230, and steps 210-230 will be repeated again. Once in the correct position, as verified in step 1230, the tissue status or type is determined in step 1240 and a further dosage of the treatment is applied to the tissue. In an alternative embodiment, only steps 1240-1260 are repeated.

In step 1270, the system checks to see if the tissue has been treated in all locations. These locations may be largely two dimensional or alternatively, three-dimensional. The system typically records the treatments applied at each location and stores this data in its memory. Processor 1108 (FIG. 9) may be configured to run software to provide a status mapping of the treatment applied to the plurality of locations on screen 1103, for example. Thus, the operator can see whether all areas of the tissue have been treated and to what degree they have been treated.

Alternatively, checking step 1270 may be performed largely manually by moving the probe from location to location and checking the status of the tissue at each location.

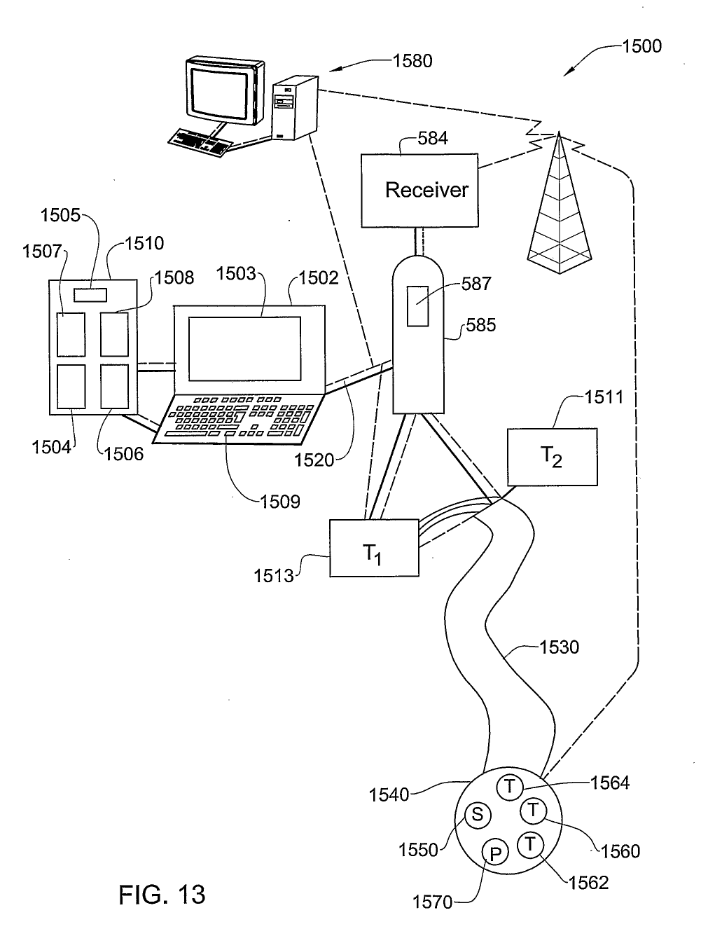

In the example of FIG. 10, only one type of treatment is applied to the tissue. In some cases, the unit dosage provide upon repeating step 1250 may be changed in response to results of step 1260, performed previously. The method of FIG. 10 may employ the system of FIG. 9 or the system of FIG. 13 (described hereinbelow). Systems similar in concept but different in construction to those described with respect to the system of FIG. 9 and the system of FIG. 13 may also be employed for performing the method of FIG. 10.

In further embodiments, the method also includes monitoring the quality of the treatment employing a quality monitoring mechanism (not shown). The quality monitoring mechanism may include, but is not limited to at least one of thermistor, thermocouple, resistivity sensor, capacitance sensor, electric field sensor, magnetic sensor, radiation sensor, or acoustic sensor.

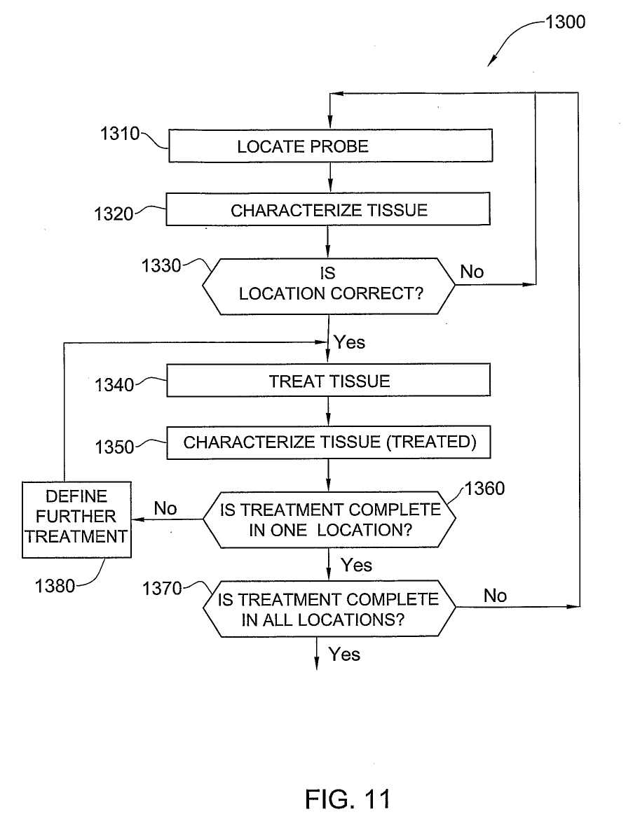

Reference is now made to FIG. 11, which is another example of a flowchart 1300 of a method for in-situ treatment of body abnormalities, in accordance with the present invention. In the method of FIG. 11, different types of treatment may be applied to the tissue.

In a locating step 1310, the probe is placed inside a lumen or on the surface of part of the body and is oriented proximal to or touching some tissue. This step may be similar to or different from step 1210 of FIG. 10.

In a characterizing step 1320, at least one sample of the tissue is characterized to determine the cell type and tissue type. For example, it is determined at this stage, whether some or all of the tissue is abnormal tissue, such as cancerous tissue. This step may be similar to or different from step 1220 of FIG. 10.

In a checking step 1330, it is determined whether the location of the probe is correct. If negative, the probe is moved to another location. The relocation of the probe may be manual, semi-manual or automatic employing for example, a two-dimensional or three-dimensional computer controlled stage, as is known in the art. There may be a computer program which controls the stage and defines the sequence of moving the probe from one location to the next. In cases, where the relocation is manual or semi-manual, the system may provide the operator with specific instructions on how and to whereto move the probe.

Additionally or alternatively, further steps of moving the stage may be introduced in response to the results of step 1330. Once the probe is relocated, steps 1320-1330 are repeated. If in step 1330, the location of the probe is correct, a tissue treating step 1340 is performed. Typically a predetermined dosage of the treatment is administered to the tissue at that location.

Thereafter, the treated tissue is characterized in a characterization step 1350. The characterization step may include qualitative and quantitative assessment of the treatment success at the specific location. If the results are unsatisfactory, one or more further types of treatment may be defined in defining step 1380, and step 1340-1360 are repeated, but using a second and or further type(s) of treatment. For example, if the first treatment is laser ablation, the second treatment may be chemotherapy and a third treatment may be ultrasound.

Once it has been determined in step 1360, that the treatment is complete in the first location, the probe is relocated in step 1310 and the treatment procedure is performed at the second location. Steps 1310-1370 or 1310-1380 are performed as many times as is required until all locations of the target tissue have been fully treated.

The method of FIG. 11 may employ the system of FIG. 9 or the system of FIG. 13 (described hereinbelow). Systems similar in concept but different in construction to those described with respect to the system of FIG. 9 and the system of FIG. 13 may also be employed for performing the method of FIG. 11.

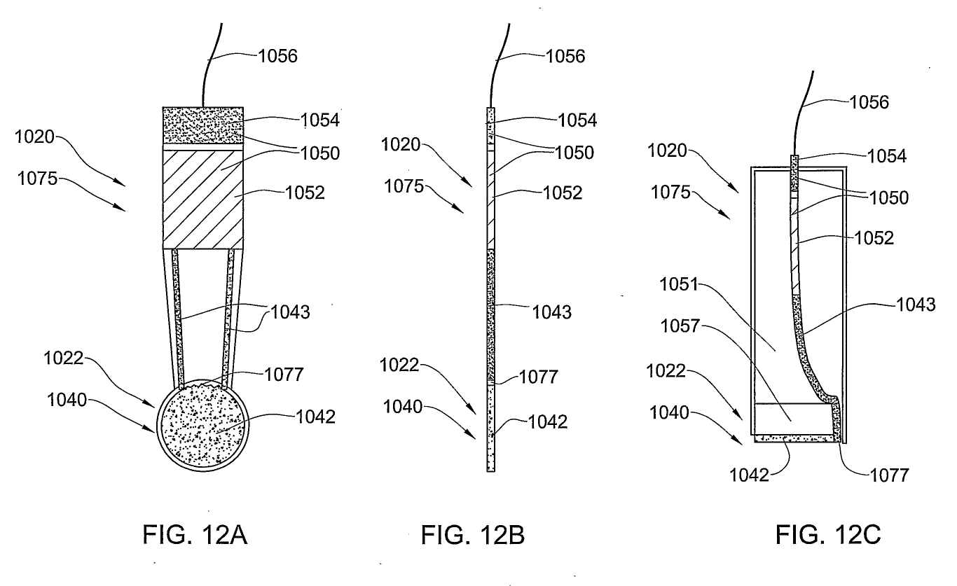

Reference is now made to FIGS. 12A-12C, which is a simplified exploded illustration of different probe examples suitable to be used in the system of FIG. 9.

Referring further to the drawings, FIG. 12A-4C schematically illustrate a sensor 1020, formed as a thin, flexible construction 1075, in accordance with an embodiment of the present invention. Preferably, sensor 1020 includes a spiral 1022 (of a thickness of about 4 microns), deposited on an insulating material 1048 (such as Kapton, of a thickness of about 100 microns), and covered with the insulating material 1048 (such as Kapton to a thickness of 50 microns), thus being essentially self-supporting.

Flexible construction 1075 is configured to bend at a line 1077, so that in operation, spiral 1022 is substantially at a right angle to the remainder of flexible construction 1075. Additionally, flexible construction 1075 is adapted for operation when inserted into a hollow housing 1074, having a top cover 1057 of polycarbon, wherein spiral 1022 forms a proximal cover over top cover 1057 of polycarbon, for forming contact or near contact with the edge 1013 of the tissue. Hollow housing 1074 essentially provides an effective cavity 1051, at the distal side of the sensor 1022.

Reference is now made to FIG. 13, which is another simplified schematic illustration of another configuration of a system 1500 for in-situ treatment of body tissue, in accordance with a preferred embodiment of the present invention.

In contrast to system 1100 of FIG. 9, in which all the parts of the system are in one integral unit in wired communication, system 1500 comprises similar major parts, but the parts are not necessarily in one unit and need not be in wired communication.

System 1500 is typically designed for in-situ treatment of a tissue at a plurality of locations of a body, so as to form an area of treatment of the tissue and to provide at least one type of treatment to the tissue. In some embodiments, at least two types of treatment are provided. In other embodiments at least three types of treatment are provided. The system includes an apparatus 1530 for proximal orientation to the tissue in the body. This apparatus is connectable to at least one external unit 1510, 1580 including inter alia a memory utility 1507, a data processing and analyzing utility 1508, data input utility 1509, a display unit 1502. The system further includes a locating apparatus 1504, and at least one treatment apparatus 1506, 1509, 1511. The external unit is in electronic communication 1520, which may be wired or wireless with apparatus 530.

Apparatus 1530 includes an active head 1540 having a sensing probe 1550 for characterizing a sample of tissue from at least one of the proximal locations. The head includes a positioning mechanism 570 for sequentially guiding the apparatus from at least one of the plurality of locations to one or more other locations of the plurality of locations. Head 1540 further includes at least one treatment probe 1560, 1562, 1564 for providing one or more type of treatments. The treatment probes include at least one active outlet (not shown) for applying at least one treatment to at least one of the plurality of proximal locations for treating the sample. Probes 1560, 1562, 1564 each include an activating mechanism (not shown) adapted to activate the at least one outlet.

The external units may or may not be in wired communication with apparatus 1530. In some cases, units 1510, 1580 may be configured for appropriate wireless communication and/or via the Internet In some embodiments, unit 1580 is a CT machine known in the art. Unit 1580 performs external imaging.

External unit 1510 includes a sensing functionality 1505 adapted to receive at least one signal from sensing probe 1550 at each location and to analyze the at least one signal so as to provide at least one output. In some cases, functionality 1505 is part of unit 1580, which may be an image analyzer, as is known in the art. One or more of treatment functionalities 1506, 1511, 1513 are adapted to receive the at least one output from unit 1580 and to provide at least one treatment to at least one of treatment probes 1560, 1562, 1564 responsive to the output. In some embodiments, the at least one treatment is provided by one or more of the treatment probes at one or more outlets (not shown). In some cases, the treatment is only provided when the activating mechanism is activated. The activating mechanism is typically activated by a signal from one or more external units 1510, 1580.

Reference is again made to FIG. 6, which is a simplified schematic partially-exploded illustration of a method for treatment of a three-dimensional abnormality inside the human body in accordance with the invention.

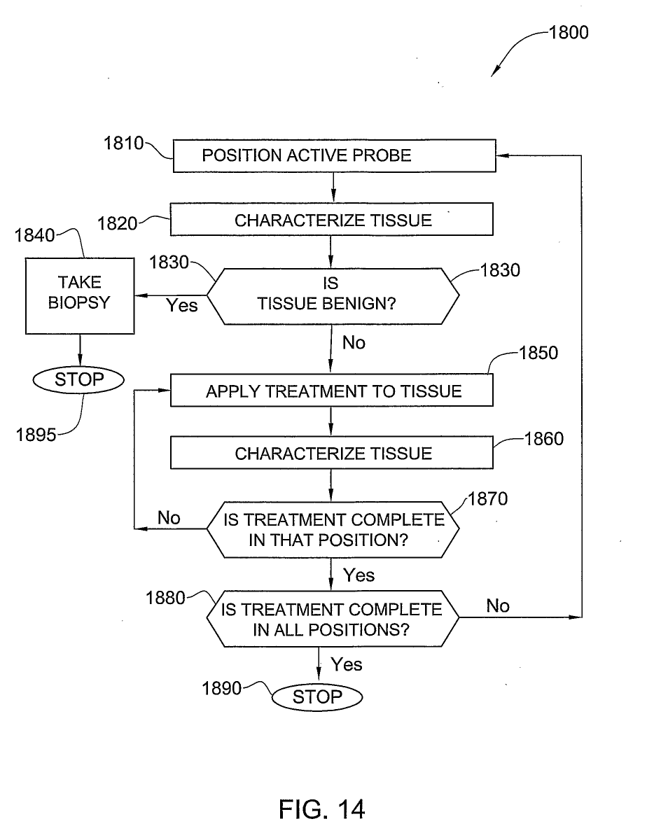

The example illustrated in FIG. 14 should not be taken to be limiting to treating the pulmonary system in a human. Rather, this invention is directed to treating both interior and exterior tissues of bodies, such as human or mammalian bodies.

In a pulmonary system, 605 of a human, there is a trachea 620 connected to two bronchi 610, 640 and two lungs 630, 632. Some abnormal tissue 612, 614, 616 was discovered in bronchus 610. It was decided, for example, to laser ablate the abnormal tissue using system 100 of FIG. 9. System 680, substantially similar to system 100 of FIG. 9, includes apparatus 600, substantially similar to apparatus 130 of FIG. 9. Apparatus 600 includes a sensing probe 602, a treatment probe 604 and a positioning probe 606. Using the positioning probe 606 and locating means 684 of system 680, apparatus 600 is moved to a first location, determined in Cartesian coordinates as x₁y₁z₁. A sample of tissue at the first location is sensed by sensing probe 602. Sensing probe sends signals to sensing module 685. The signals are processed by processor 688. Processor 688 provides an output to screen 603, indicative of that the tissue at the first location is normal. The operator or the system now moves apparatus 600 to a second location x₂y₂z₁. At the second location, a sample of the tissue is analyzed as for the tissue at the first position and the tissue is found to be normal. No treatment is provided at positions where the tissue is normal.

The apparatus is now moved to a third position x₁y₁z₂. A sample of tissue at the third location is sensed by sensing probe 602. Sensing probe sends signals to sensing module 685; the signals are processed by processor 688; and corresponding output is displayed, being indicative of that the tissue at the third location is abnormal. In an optional verification step, another type and/or another set of signals is relayed by the sensing probe to the processor, and the tissue type at the third position is characterized and/or verified. The type, duration, quantity and quality of the treatment may be determined at this stage.

Treatment functionality 686 activates a laser beam, such as a Yag-Niobium laser via apparatus 600 to treatment probe. The treatment probe provides the laser ablation treatment for a predetermined period of time. The sensing probe then senses the treated tissue and sends signals to the processor. In accordance with the so-provided (displayed) output, the operator can determine, what, if any, further treatment is required at the third location. Alternatively, the determination may be made by the processor, based on a set of predetermined rules.

If the treatment at the third location is complete, the apparatus is moved to a fourth position. The sensing, treating and positioning steps are repeated until all of the abnormal tissue 612, 614, 616 and 618 has been treated. Thereafter, the system may perform a full scan at all locations to verify that all the abnormal tissue has been treated.

Reference is again made to FIG. 8, which is a schematic illustration of an example of a method for treatment of a substantially two-dimensional abnormality of the skin, in accordance with the invention.

Using the system of FIG. 9 or of FIG. 13, an abnormality on the skin surface may be treated. Apparatus 700 is shown being configured substantially similar to apparatus 130 of FIG. 9. Apparatus 700 is moved around a substantially two dimensional surface areas of abnormal tissue 712, 714 which are treated by the method of either FIG. 10 or FIG. 11.

In some embodiments, the apparatus is designed to treat tissue inside body lumens, such as inside a lung bronchus. The particular design features associated with treating a body lumen are shown in FIG. 6. In some preferred embodiments, the apparatus is designed to treat tissue intra-operatively. The particular design features associated with intra-operative treatment are shown in FIG. 7.

In some other preferred embodiments, the apparatus is designed to treat skin or an external surface of the body. The particular design features associated with treating skin are shown in FIG. 8. For example a photodynamic applicator such as a laser or high intensity lamp with a filter, as are known in the art, may be used as the treatment probe.

Systems for physical treatment are exemplified by as electrosurgery, cryo-therapy, RF ablation examples. Systems for chemical treatment include, but are not limited to applying a. toxin, or drug examples. Systems for biological treatment include, but are not limited to DNA therapy, viral therapy, enzyme therapy, vial therapy examples. In some embodiments, the sensing apparatus includes a localization mechanism, which is computer controlled with a feedback mechanism examples.

Reference is now made to FIG. 14, which is a flowchart 1800 of a method for in-situ characterization and treatment of a lesion, in accordance with a preferred embodiment of the present invention.

In a positioning step 1810, an apparatus, such as apparatus 1130 of FIG. 9, including active head 1140 having sensing probe 1150 or any other active probe, is placed inside a lumen or on the surface of part of the body and is oriented proximal to or touching some tissue. This step may be similar to or different from step 1210 of FIG. 10.

The positioning step of the probe may be manual, semi-manual or automatic employing for example, a two-dimensional or three-dimensional computer controlled stage, as is known in the art. There may be a computer program which controls the stage and defines the sequence of moving the probe from one location to the next. In cases, where the relocation is manual or semi-manual, the system may provide the operator with specific instructions on how and to whereto move the probe.

In a characterizing step 1820, at least one sample of the tissue is characterized to determine the cell type and tissue type. For example, it is determined at this stage, whether some or all of the tissue is abnormal tissue, such as cancerous tissue. This step may be similar to or different from step 1220 of FIG. 10. The characterized “abnormal” tissue is defined as being different to normal tissue. The abnormality may be due to a difference measured relative to the “normal tissue. The difference may be one or more of a physical difference, such as density or fluidity, a chemical difference, such as acidity, a biological difference, such as enzymatic activity, or a genetic difference, characterized by a gene expression activity. The difference may be defined on-line, off-line or by a combination thereof. The system or systems employed for characterizing the difference or differences may be any one or more of the systems described herein, or may also employ one or more other systems known in the art.

In a checking step 1830, it is determined whether the characterized “abnormal” tissue is benign. This step may be performed off-line by a pathologist in a lab, or online by a tissue characterization apparatus, such as the system of FIG. 9. If the tissue is benign, some or all of the tissue may be removed surgically in a biopsy taking step 1840. Once the biopsy has been taken, the process is completed in step 1895 for treating that specific tissue.

If in step 1830, the tissue is characterized as not being benign, one or more treatments are applied to the tissue in step 1850. It should be understood that steps 1820 and 1830 may comprise several substeps, in which the type of tissue may be further characterized. For example, step 1820 may include sub-steps to characterize the size and location of the abnormal tissue relative to the normal tissue. Step 1830 may comprise sub-steps to further determine the sub-type of the abnormal tissue, such as cancer type, tumor type, size, age, density, relative location. Once the non-benign tissue is fully characterized and located, it is treated in step 1850. Typically a predetermined dosage of the treatment is administered to the tissue at that location. The treatment administered may be similar or different to that of step 1250 of FIG. 10 hereinabove.

In a characterization step 1860, the treated tissue is characterized to check the success of the treatment step. For example, if the cells of the treated tissue were laser ablated in step 1850, step 1860 is performed to check what percentage of cells were successfully treated and if any of the cells were overlooked in the treatment step.

In a checking step 1870, the results of characterization step 1860 are analyzed to see whether any further treatment is required for that specific tissue. The further treatment may be related to location and or to extent of the treatment. For example, a first result of step 1860 could be that 85% of the cells were successfully treated, but 15% of the cells were not treated. Then the result of step 870 would be negative and the treatment would be repeated for the 15% of the cells. A second result of step 1860 could be that all the cells were almost fully ablated, say 70%, then the result of step 1870 would be negative and the treatment would be repeated for all the cells until 100% treatment was applied for all the cells in the repeat of step 1850.

If the result of step 1870 is that all the cells of the tissue were sufficiently treated, then probe is positioned at the next location in a repeat of step 1810 and the treatment procedure is performed at the second location.

In checking step 1880, it is determined whether tissue at all of the proximal locations, whether three-dimensional or two dimensional locations have been fully treated. If not, the probe is moved to the next location in step 1810 until tissue at all of the locations has been fully treated. Thus, steps 1810-1880 are performed as many times as is required until all locations of the target tissue have been fully treated.

The method of FIG. 10 may employ the system of FIG. 9 or the system of FIG. 13 (described hereinbelow). Systems similar in concept but different in construction to those described with respect to the system of FIG. 9 and the system of FIG. 13 may also be employed for performing the method of FIG. 14.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art.

1. A system for analysis and treatment of a tissue site comprising:
(a) a device having a probe unit comprising:
i. one or more tissue sensing and monitoring probes configured to measure one or more parameters indicative of one or more states of the tissue site; and
ii. one or more tissue treatment probes configured to deliver one or more treatments to the tissue site;
(b) a controller comprising a processor configured to:
i. receive and process signals from the one or more tissue sensing and monitoring probes and determine whether the probe unit is located at the tissue site to be treated;
ii. activate the one or more of the treatment probes and the one or more sensing and monitoring probes to monitor one or more states of the tissue site while delivering the one or more treatments to the tissue site; and
iii. as the treatment proceeds, receive and analyze signals from the one or more sensing and monitoring probes, indicative of one or more measurements obtained by the one or more tissue sensing and monitoring probes, determine whether the monitored tissue parameters correspond to the required treatment and determine whether the treatment parameters need to be modified or whether predetermined treatment targets have been achieved.

2. The system according to claim 1, wherein said probe unit comprises the same one or more probes for carrying out said sensing prior to treatment and said monitoring during the treatment.

3. The system according to claim 1, wherein said probe unit comprises one or more probes for the sensing prior to treatment and one or more probes for the monitoring during the treatment.

4. The system according to claim 1, wherein the device is in the form of a catheter.

5. The system according to claim 1, wherein the at lease one tissue sensing and monitoring probe is selected from the following:
(a) an optical sensor;
(b) an X-ray sensor;
(c) a radiofrequency (RF) sensor;
(d) a microwave (MW) sensor;
(e) an infrared thermography sensor;
(f) an ultrasound sensor;
(g) a magnetic resonance (MR) sensor;
(h) an impedance sensor;
(i) a resistivity sensor;
(j) capacitance sensor;
(k) an electric field sensor;
(l) a magnetic sensor;
(m) a radiation sensor;
(n) an acoustic sensor;
(o) a thermistor or temperature sensor;
(p) a thermocouple;
(q) a biosensor;
(r) a chemical sensor;
(s) a radioactive-emission sensor;
(t) a mechanical sensor; and
(u) an endoscope.

6. The system according to claim 1, wherein at least one treatment probe is selected from the following:
(a) an injector;
(b) a bombardment device;
(c) a device for localized surgery;
(d) a device for resection, cryosurgery, or laser surgery;
(e) a device for ablation;
(f) an ultrasound device;
(g) a radiofrequency (RF) device;
(h) a biopsy device;
(i) a microwave (MW) device; and
(j) a dispensing device.

7. The system according to claim 1 wherein the probe unit is configured to be detectable by an imaging system.

8. The system according to claim 1 further comprising an imaging system.

9. The system according to claim 8 wherein the imaging system is selected from the group:
(a) An X-ray imaging system;
(b) an ultrasound imaging system; and
(c) a magnetic resonance imaging (MRI) system.

10. The system according to claim 1 comprising three or more tissue sensing probes or tissue monitoring probes positioned at the distal end of the catheter in a curved array.

11. The system according to claim 10 wherein the processor is further configured to analyze signals from the three or more tissue sensing probes to obtain three-dimensional information relating to the tissue site.

12. The system according to claim 4 wherein the catheter is a cannula having one or more working channels configured to receive a tissue sensing probe, a tissue monitoring probe or a treatment probe.

13. The system according to claim 1 wherein the control unit further comprises a monitor.

14. The system according to claim 1 further comprising a positioning mechanism for guiding the probe unit from at least one of a plurality of locations to one or more other locations of the plurality of locations.

15. The system according to claim 1 wherein the processor is contained in a handle.

16. The system according to claim 1, wherein the tissue sensing probe comprises a resonating element, formed as a conductive structure, and configured to be placed at an edge of the tissue site without penetrating into the tissue.

17. A device for analysis and treatment of a tissue site comprising:
(a) a probe unit comprising
i. one or more tissue sensing and monitoring probes configured to measure one or more parameters indicative of one or more states of the tissue site; and
ii. one or more tissue treatment probes configured to deliver a treatment to the tissue site.

18. The device according to claim 17, wherein the device is selected from the following:
an extracorporeal device, an intracorporeal device, a device adapted for use on a portion of subcutaneous tissue, and a device adapted for use on a portion of an intracorporeal tissue during an open surgery.

19. The device according to claim 18, wherein the intracorporeal device is selected from the following:
a device configured for minimally invasive surgery; a device configured for insertion via a trocar valve; a device configured for insertion via body orifice to a body lumen for use on a portion of inner lumen wall, a device adapted for percutaneous insertion to a body lumen for use on a portion of inner lumen wall; a device adapted for insertion via body orifice to a body lumen, for further penetrating the lumen for use on a portion of an intracorporeal tissue outside the lumen.

20. A method for analysis and treatment of a tissue site comprising:
(a) delivering to the tissue site a probe unit comprising one or more treatment probes, one or more tissue sensing and monitoring probes
(b) measuring one or more values of one or more parameters indicative of one or more states of said tissue site for determining whether the probe unit is located at a tissue site requiring treatment prior to activating the one or more treatment probes.
(c) activating the one or more treatment probes to deliver the one or more determined treatments to the tissue site;
(d) receiving signals from the one or more tissue sensing and monitoring probes during delivery of the treatment indicative of one or more measurements obtained by the one or more tissue monitoring probes; and
(e) determining whether the monitored tissue parameters correspond to the required treatment and determine whether the treatment parameters need to be modified or whether predetermined treatment targets have been achieved.

21. The method according to claim 20 wherein the tissue site is located on a body surface.

22. The method according to claim 20 wherein the tissue site is located at an internal body site.

23. The method according to claim 20 wherein one or more of the tissue sensing probes or the tissue treatment probes are inserted into the body through a body orifice.

24. The method according to claim 20 wherein one or more of the tissue monitoring probes or the tissue treatment probes are inserted into the body through an incision in the skin.

25. The method according to claim 20 wherein one or more of the tissue sensing probes and the treatment probes are located at a distal end of a catheter shaft.

26. The method according to claim 20 wherein the tip of the catheter shaft is detectable in an imaging system.

27. The method according to claim 20 further comprising determining whether one or more of the probes is located at the tissue site.

28. The method according to claim 20 wherein one or more of the measurements obtained by the one or more tissue sensing probes at the tissue site is a physical measurement.

29. The method according to claim 28 wherein the physical measurement is a density measurement or a fluidity measurement.

30. The method according to claim 20 wherein one or more of the measurements obtained by the one or more tissue sensing probes at the tissue site is a chemical measurement.

31. The method according to claim 30 wherein the chemical measurement is an acidity measurement.

32. The method according to claim 20 wherein one or more of the measurements obtained by the one or more tissue sensing probes at the tissue site is a biological measurement.

33. The method according to claim 32 wherein the biological measurement is an enzymatic activity measurement, or a measurement of a level of gene expression.

34. The method according to claim 20 further comprising classifying the tissue at the tissue site.

35. The method according to claim 34 wherein tissue is classified into a normal class or an abnormal class.

36. The method according to claim 35 wherein an abnormal class is a malignant state or a precancerous state.

37. The method according to claim 35 wherein an abnormal class is a pathological state.

38. The method according to claim 34 further comprising displaying the classification generated on a monitor.

39. The method according to claim 38 wherein the classification is displayed graphically, numerically or textually.

40. The method according to claim 20 further comprising obtaining a biopsy at the tissue site.

41. The method according to claim 20 wherein the tissue site is located adjacent to a body lumen and the step of delivering a probe unit the tissue site comprises
(a) delivering the probe unit to the body lumen; and
(b) passing the probe unit through a wall of the lumen to the body site.
